SEQUENCE LISTING (8 sequences)

SEQ ID NO: 1 - DNA, 20 bp, Homo sapiens
agcacttagtagctgtggag

SEQ ID NO: 2 - DNA, 21 bp, Homo sapiens
ctgtggaaccttgctaccacc

SEQ ID NO: 3 - DNA, 17 bp, Homo sapiens
ggtggtttcccaacaaa

SEQ ID NO: 4 - DNA, 19 bp, Homo sapiens
gccaggtgtggtaactgct

SEQ ID NO: 5 - DNA, 21 bp, Homo sapiens
gtttgacggcaggaagaatct

SEQ ID NO: 6 - DNA, 21 bp, Homo sapiens
aggacacccacttgatggaca

SEQ ID NO: 7 - DNA, 20 bp, Homo sapiens
cggctaccacatccaaggaa

SEQ ID NO: 8 - DNA, 18 bp, Homo sapiens
gctggaattaccgcggct

(19) United States
(12) Patent Application Publication
(10) Pub. No.: US 2019/0367918 A1
(43) Pub. Date: Dec. 5, 2019

(54) COMPOSITIONS AND METHODS FOR DELIVERING MICRORNA

(71) Applicant: NeOnc Technologies, Inc., Los Angeles, CA (US)

(72) Inventors: Florence M. Hofman, Venice, CA (US); Raquel M.S. Ferreira, Braga (PT); Steven L. Giannotta, Pasadena, CA (US); Thomas C. Chen, La Cañada Flintridge, CA (US)

(21) Appl. No.: 16/432,532

(22) Filed: Jun. 5, 2019

Related U.S. Application Data
(63) Continuation of application No. 16/169,606, filed on Oct. 24, 2018.
Application No. 16/169,606 is a continuation of application No. 15/122,381, filed on Aug. 29, 2016, now abandoned.
(60) Provisional application No. 61/952,063, filed on Mar. 12, 2014.

(51) Int. Cl. C12N 15/113; C12N 15/11; A61K 31/713; A61K 38/46; A61K 45/06

(52) U.S. Cl. CPC C12N 15/113; C12N 15/111; A61K 31/713; C12N 2320/32; A61K 45/06; C12Y 301/26; C12N 2310/141; A61K 38/465

(57) ABSTRACT

The invention relates to compositions, methods and kits for using Argonaute-2 (Ago-2) as a systemic carrier to deliver a miRNA to an endothelial cell. The invention also relates to compositions, methods and kits for inhibiting angiogenesis and/or treating a condition by using Ago-2 as a systemic carrier to deliver a miRNA to an endothelial cell. The condition includes but is not limited to brain vascular diseases and brain tumors.

### FIELD OF THE INVENTION

The invention relates to the use of Argonaute-2 (Ago-2) as a systemic carrier to deliver a microRNA (miRNA) to a cell, particularly a brain endothelial cell. The invention also relates to compositions, methods and kits for inhibiting angiogenesis and/or treating a condition by using Ago-2 as a systemic carrier to deliver a miRNA to an endothelial cell. The condition includes but is not limited to cerebrovascular disorders and brain tumors.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The systemic delivery of miRNA without transfection reagents (naked delivery) has been successfully accomplished for the reduction of tumor metastasis in the mouse liver, by showing that naked miRNA can be internalized by tumor cells. In addition, intravenous injection of naked miRNA was shown to also enter virally infected liver cells. These applications were focused solely on the systemic delivery of miRNA into highly vascularized liver as the target organ, and not relevant to the neurovasculature.

Cerebral Arteriovenous Malformations (AVM) are brain vascular lesions comprising an abnormal tangle of vessels (nidus), in which arteries and veins are directly connected without an intervening capillary system. AVM affect approximately 300,000 people in the USA and can lead to serious neurological symptoms or death. Current medical treatments are highly invasive and can pose significant risks to nearby brain structures that regulate speech, movement and sensory processing, highlighting the importance of developing more efficacious and safer therapies.

### SUMMARY OF THE INVENTION

Human AVM-derived brain endothelial cells (AVM-BEC) have distinct and abnormal characteristics compared to normal BEC. Namely, AVM-BEC proliferate more rapidly, migrate faster, and produce aberrant vessel-like structures as compared to normal vasculature. AVM-BEC also express low levels of a key regulator of angiogenesis, thrombospondin-1 (TSP-1). These abnormal features are ameliorated with microRNA-18a (miR-18a) treatment. MiRNAs are small non-coding RNAs that inhibit gene expression by inducing cleavage or translational repression of messenger RNA (mRNA). Specifically, miR-18a inhibited TSP-1 transcriptional repressor, Inhibitor of DNA-binding protein-1 (Id-1), leading to increased TSP-1 levels and decreased vascular endothelial growth factor (VEGF)-A and VEGF-D secretion. miR-18a also regulated cell proliferation and improved tubule formation efficiency. Importantly, these effects were obtained with miRNA alone (naked delivery), in the absence of traditional transfection reagents, like lipofectamine, which cannot be used in vivo due to induced toxicity. Naked miRNAs have been shown to form complexes with circulating RNA-binding proteins, such as argonaute-2 (Ago-2), a member of the Argonaute protein family, which also includes Ago-1, Ago-3 and Ago-4. In human cells, Ago-2 takes part in the RNA-induced silencing complex (RISC) to promote endonucleolytic cleavage of mRNA.

In this application, we show that AVM-BEC release Ago-2, which can be used to enhance the entry of extracellular miR-18a into brain endothelial cells. In vitro studies show that Ago-2 in combination with miR-18a is functional and able to stimulate TSP-1 production. Furthermore, miR-18a in combination with Ago-2 can be delivered in vivo by intravenous administration, resulting in increased circulating serum TSP-1 and decreased VEGF-A. Thus Ago-2 may be used to decrease angiogenic activity in brain endothelial cells, making Ago-2 a biocompatible miRNA-delivery platform suitable for treating neurovascular diseases and brain tumors.

Various embodiments of the present invention provide a method of delivering a miRNA to a cell. The method may comprise or may consist of: providing the miRNA and an Ago-2 or a variant thereof; and contacting the cell with the miRNA and the Ago-2 or the variant thereof, thereby delivering the mRNA to the cell. In some embodiments, the miRNA and the Ago-2 or the variant thereof are provided in one composition. In other embodiments, the miRNA and the Ago-2 or the variant thereof are provided in separate compositions. Various embodiments of the present invention provide a kit for delivering a miRNA to a cell. The kit may comprise or may consist of a quantity of a miRNA; a quantity of an Ago-2 or a variant thereof; and instructions for using the Ago-2 or the variant thereof to deliver the miRNA.

Various embodiments of the present invention provide a method of delivering a miRNA to a cell. The method may comprise or may consist of: providing the miRNA and an Ago-2 or a variant thereof; mixing the miRNA with the Ago-2 or the variant thereof; and contacting the cell with the mixture of the miRNA and the Ago-2 or the variant thereof, thereby delivering the mRNA to the cell. In various embodiments, the miRNA and the Ago-2 or the variant thereof form a ribonucleoprotein complex in the mixture.

Various embodiments of the present invention provide a method of inhibiting or suppressing angiogenesis in a subject. The method may comprise or may consist of: providing a miRNA and an Argonaute-2 (Ago-2) or a variant thereof; administering a therapeutically effective amount of the miRNA and the Ago-2 or the variant thereof to the subject, thereby inhibiting or suppressing angiogenesis in the subject. In some embodiments, the miRNA and the Ago-2 or the variant thereof are provided in one composition. In other embodiments, the miRNA and the Ago-2 or the variant thereof are provided in separate compositions. Various embodiments of the present invention provide a kit for inhibiting or suppressing angiogenesis. The kit may comprise or may consist of a quantity of a miRNA; a quantity of an Argonaute-2 (Ago-2) or a variant thereof; and instructions for using the miRNA and the Ago-2 or the variant thereof to inhibit or suppress angiogenesis. In some embodiment, the miRNA is capable of inhibiting or suppressing angiogenesis.

Various embodiments of the present invention provide a method of inhibiting or suppressing angiogenesis in a subject. The method may comprise or may consist of: providing a miRNA and an Ago-2 or a variant thereof; mixing the miRNA with the Ago-2 or the variant thereof; and administering a therapeutically effective amount of the mixture to the subject, thereby inhibiting or suppressing angiogenesis in the subject. In various embodiments, the miRNA and the Ago-2 or the variant thereof form a ribonucleoprotein complex in the mixture. In some embodiment, the miRNA is capable of inhibiting or suppressing angiogenesis.

Various embodiments of the present invention provide a method of promoting angiogenesis in a subject. The method may comprise or may consist of: providing a miRNA and an Argonaute-2 (Ago-2) or a variant thereof; administering a therapeutically effective amount of the miRNA and the Ago-2 or the variant thereof to the subject, thereby promoting angiogenesis in the subject. In some embodiments, the miRNA and the Ago-2 or the variant thereof are provided in one composition. In other embodiments, the miRNA and the Ago-2 or the variant thereof are provided in separate compositions. Various embodiments of the present invention provide a kit for promoting angiogenesis. The kit may comprise or may consist of a quantity of a miRNA; a quantity of an Argonaute-2 (Ago-2) or a variant thereof; and instructions for using the miRNA and the Ago-2 or the variant thereof to promote angiogenesis. In some embodiments, the miRNA is capable of promoting angiogenesis.

Various embodiments of the present invention provide a method of promoting angiogenesis in a subject. The method may comprise or may consist of: providing a miRNA and an Ago-2 or a variant thereof; mixing the miRNA with the Ago-2 or the variant thereof; and administering a therapeutically effective amount of the mixture to the subject, thereby promoting angiogenesis in the subject. In various embodiments, the miRNA and the Ago-2 or the variant thereof form a ribonucleoprotein complex in the mixture. In some embodiments, the miRNA is capable of promoting angiogenesis.

Various embodiments of the present invention provide a method of treating, preventing, reducing the likelihood of having, reducing the severity of and/or slowing the progression of a condition in a subject. The method may comprise or may consist of: providing a miRNA and an Argonaute-2 (Ago-2) or a variant thereof; administering a therapeutically effective amount of the miRNA and the Ago-2 or the variant thereof to the subject, thereby of treating, preventing, reducing the likelihood of having, reducing the severity of and/or slowing the progression of a condition in the subject. In some embodiments, the miRNA and the Ago-2 or the variant thereof are provided in one composition. In other embodiments, the miRNA and the Ago-2 or the variant thereof are provided in separate compositions. Various embodiments of the present invention provide a kit for treating, preventing. reducing the likelihood of having, reducing the severity of and/or slowing the progression of a condition. The kit may comprise or may consist of a quantity of a miRNA: a quantity of an Argonaute-2 (Ago-2) or a variant thereof: and instructions for using the miRNA and the Ago-2 or the variant thereof to treat, prevent, reduce the likelihood of having, reduce the severity of and/or slow the progression of the condition in the subject.

Various embodiments of the present invention provide a method of treating, preventing, reducing the likelihood of having, reducing the severity of and/or slowing the progression of a condition in a subject. The method may comprise or may consist of: providing a miRNA and an Ago-2 or a variant thereof; mixing the miRNA and the Ago-2 or the variant thereof; and administering a therapeutically effective amount of the mixture to the subject, thereby treating, preventing, reducing the likelihood of having, reducing the severity of and/or slowing the progression of the condition in the subject. In various embodiments, the miRNA and the Ago-2 or the variant thereof form a ribonucleoprotein complex in the mixture.

In one embodiment, the miRNA and the Ago-2 or the variant thereof may be provided in one composition. In another embodiment, the miRNA and the Ago-2 or the variant thereof may be provided in two separate compositions. In various embodiments, the miRNA is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L. In various embodiments, the miRNA is administered intratumorally, intracranially, intraventricularly, intrathecally, epidurally, intradurally, intravascularly, intravenously, intraarterially, intramuscularly, subcutaneously, intraperitoneally, intranasally, or orally. In various embodiments, the miRNA is administered once, twice, three or more times. In various embodiments, the mixture is administered 1-3 times per day, 1-7 times per week, or 1-9 times per month. In various embodiments, the miRNA is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In various embodiments, the Ago-2 or the variant thereof is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L. In various embodiments, the Ago-2 or the variant thereof is administered intratumorally, intracranially, intraventricularly, intrathecally, epidurally, intradurally, intravascularly, intravenously, intraarterially, intramuscularly, subcutaneously, intraperitoncally, intranasally, or orally. In various embodiments, the Ago-2 or the variant thereof is administered once, twice, three or more times. In various embodiments, the Ago-2 or the variant thereof is administered 1-3 times per day, 1-7 times per week, or 1-9 times per month. In various embodiments, the Ago-2 or the variant thereof is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years.

Various methods described herein may further comprise providing and administering a therapeutically effective amount of an anti-angiogenic drug to the subject. Various kits described herein may further comprise a quantity of an anti-angiogenic drug. Various methods described herein may further comprise providing and administering a therapeutically effective amount of a chemotherapeutic agent to the subject. Various kits described herein may further comprise a quantity of a chemotherapeutic agent.

Various embodiments of the present invention provide a composition. The composition may comprise or may consist of a miRNA and an Ago-2 or a variant thereof. In accordance with the present invention, the composition may be used for delivering the miRNA to a cell, inhibiting angiogenesis, promoting angiogenesis, and/or treating, preventing, reducing the likelihood of having, reducing the severity of and/or slowing the progression of a condition in a subject.

Various embodiments of the present invention provide a composition. The composition may comprise or may consist of a ribonucleoprotein complex of a miRNA and an Ago-2 or a variant thereof. In accordance with the present invention, the composition may be used for delivering the miRNA to a cell, inhibiting angiogenesis, promoting angiogenesis and/or treating, preventing, reducing the likelihood of having, reducing the severity of and/or slowing the progression of a condition in a subject.

In various embodiments, the subject is a human. In various embodiments, the miRNA is miR-18a or miR-128a. In some embodiment, the miRNA is capable of inhibiting or suppressing angiogenesis (e.g., miR-92, miR-92a, miR-221/22). In other embodiment, the miRNA is capable of promoting angiogenesis (e.g., miR-296, miR-126, mir-210, miR-130).

Various compositions described herein may be formulated for intratumoral, intracranial, intraventricular, intrathecal, epidural, intradural, intravascular, intravenous, intraarterial, intramuscular, subcutaneous, intraperitoneal, intranasal, or oral administration. Various compositions described herein may further comprise an anti-angiogenic drug. Various compositions described herein may further comprise a chemotherapeutic agent. Various compositions described herein may further comprise a pharmaceutically acceptable excipient. Various compositions described herein may further comprise a pharmaceutically acceptable carrier.

In accordance with the present invention, examples of anti-angiogenic drugs include but are not limited to Genentech/Roche (Bevacizumab/Avastin®), Bayer and Onyx Pharmaceuticals (sorafenib/Nexavar®), Pfizer (sutinib/Sutentt), GlaxoSmithKline (pazopanib/Votrient®), Novartis (everolimus/Affinitor®), Celgene (pomalidomide/Pomalyst®) and Ipsen and Active Biotech (tasquinimod/ABR-215050, CID 54682876).

In accordance with the present invention, examples of the chemotherapeutic agent include but are not limited to Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin. Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panirumab, Pemetrexed, Rituximab, Tafluposide, Teniposide. Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mcrcaptopurine (6-MP), Cladribine, Clofarabine, Floxuridine, Fludarabine, Pentostatin, Mitomycin, ixabepilone, Estramustine, prednisone, methylprednisolone, dexamethasonc or a combination thereof.

Various compositions, methods and kits of the present invention find utility in the treatment of various conditions, including but not limited to neurovascular disease, brain vascular disease, cerebra arteriovenous malformation (AMV), stroke, tumor or cancer, brain tumor, glioma, glioblastoma, and glioblastoma multiform (GBM).

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 shows, in accordance with various embodiments of the invention, that AVM-BEC-conditioned media (AVM-BEC-CM) potentiates miR-18a internalization. A) AVM-BEC and control BEC were analyzed for intracellular miR-18a levels using qPCR. Control BEC were used as baseline (n=3; **p<0.01). B) miR-18a (40 nmol/L) in combination with AVM-BEC-CM (black bars) or fresh culture media (white bars) was added to AVM-BEC and tested for intracellular miR-18a after 5, 10, 30 minutes, and 24 hours. AVM-BEC-CM enhanced miR-18a entry up to 30 minutes (n=3-4; *p<0.05; **p<0.01). C) Control BEC were treated with miR-18a (40 nmol/L) in combination with AVM-BEC-CM (black bar), control BEC-CM (gray bar) or fresh media (white bar). Intracellular miR-18a was analyzed (qPCR) after 30 minutes incubation; AVM-BEC-CM potentiated miR-18a internalization by control BEC (n=3; **p<0.01). D) Control BEC were treated with miR-18a (40 nmol/L) and in the presence of serial diluted AVM-BEC-CM (diagonal line bars), demonstrating that progressively diluted AVM-BEC-CM loses its ability to enhance miR-18a internalization (n=3, **p<0.01, ***p<0.001). Dotted line represents miR-18a uptake by control BEC in the presence of fresh media.

FIG. 2 shows, in accordance with various embodiments of the invention, that Ago-2 is highly expressed by AVM-BEC. A) Basal expression of RNA-binding proteins (NPM, nucleophosmin-1; NCL, nucleolin; Ago-2, argonaute-2) in AVM-BEC and control BEC were analyzed by qPCR. Increased levels of NCL and Ago-2 in AVM-BEC as compared to control BEC were detected (n=3; **p<0.01; ***p<0.001). B) AVM-BEC were treated with siAgo (30-75 nmol/L), scrambled siAgo (50-75 nmol/L) and lipofectamine (2 ug/ml) and Ago-2 protein levels were analyzed by Western blotting. siAgo-2 (75 nmol/L) decreased approximately 50%/o of intracellular Ago-2 protein content (n=3; *p<0.05). A representative image depicting the effects of siAgo-2 (75 nmol/L) alone and in the presence of lipofectamine (2 μg/ml) is shown below.

FIG. 3 shows, in accordance with various embodiments of the invention, that Ago-2 silencing compromises miR-18a entry. A) Intracellular miR-18a detection in AVM-BEC, control BEC, tumor-derived endothelial cells (TuBEC), human umbilical vein endothelial cells (HUVEC), human microvascular endothelial cells (HMEC) and astrocytes treated with miR-18a (40 nmol/L) in the presence of siAgo-2-AVM-BEC-CM (dotted bars) or AVM-BEC-CM (black bars). Ago-2 silencing decreased miR-18 entry in AVM-BEC, control BEC and TuBEC (n=3; *p<0.05; ***p<0.001). B) Intracellular detection of miR-18a in control BEC (qPCR) after treating cells for 30 minutes with different concentrations of Ago-2 (0.01-4 nmol/L) in combination with miR-18a (40 nmol/L) showed that higher concentrations of Ago-2 (up to 00.4 nmol/L) increased miR-18a detection (n=3). Dotted line represents miR-18a uptake by control BEC in the presence of AVM-BEC-CM. C) Analysis of intracellular miR-18a (qPCR) showed that miR-18a (40 nmol/L) in combination with Ago-2 (0.4 nmol/L) (for 5, 30, 120 and 1440 minutes) was more resistant to degradation than miR-18a alone; maximum effect was observed at 120 minutes (n=3; **p<0.01). D) AVM-BEC and control BEC were exposed to miR-18a in combination with siAgo-2-AVM-BEC-CM or AVM-BEC-CM. Ago-2 staining (red) showed that cells exposed to AVM-BEC-CM increased Ago-2 detection in control BEC when treated with miR-18a (40 nmol/L) (n=3, **p<0.01). Nuclear staining is shown in blue.

FIG. 4 shows, in accordance with various embodiments of the invention, that facilitated transport is involved in miR-18a delivery. A) AVM-BEC and control BEC were treated with AVM-BEC-CM plus miR-18a (40 nmol/L) at 4° C. and 37° C. for 30 minutes. Intracellular miR-18a was measured using qPCR as described previously, showing that at 4° C. miRNA entry was only minimally compromised (n=3). B) The distribution of Ago-2 (red) was identified using immunocytochemistry. At 4° C. untreated AVM-BEC expressed high levels of intracellular Ago-2 (i) compared to untreated control BEC (ii). When control BEC were treated with AVM-BEC-CM plus miR-18a at 4° C., Ago-2 staining was apparent and associated with the cell membrane (iii; white arrows) (n=3). Blue staining denotes nuclear staining. C) The formation of a ribonucleoprotein complex between Ago-2 and miR-18a was determined by immunoprecipitation and immunoblotting (left panel) and qPCR (right panel). Ago-2 was detected only in the two fractions in contact with anti-Ago-2, as expected. Only the fraction with both Ago-2 and miR-18, but not miR-18a alone, led to the detection of miR-18a by qPCR. Rabbit IgG served as the isotypic control.

FIG. 5 shows, in accordance with various embodiments of the invention, that Ago-2 silencing decreases miR-18a-induced TSP-1 secretion. A) AVM-BEC were treated with siAgo-2 (75 nM) followed by miR-18a treatment (40 nmol/L) and cell supernatants tested for TSP-1 (n=4; *p<0.05). B) Control BEC were treated with varying concentrations of the miR-18a inhibitory sequence, antagomir (40-120 nmol/L). Antagomir treatment (80 nmol/L) significantly decreased TSP-1 levels (n=4; **p<0.01).

FIG. 6 shows, in accordance with various embodiments of the invention, that Co-treatment of miR-18a and Ago-2 in vivo “normalizes” TSP-1 and VEGF-A plasma levels. A) Athymic nude mice were implanted with glioma cells intracranially. After 3 days, animals were treated intravenously with vehicle, miR-18a plus Ago-2, miR-18a alone or Ago-2 alone every 48 hours for three cycles. Subsequently. plasma was tested for TSP-1 (A) and VEGF-A (B). miR-18a and Ago-2 combination treatment caused the most significant increase of TSP-1 levels (n=5; *p<0.05; **p<0.01, ***p<0.001), and reduction of VEGF-A levels (n=5; *p<0.05). Control plasma (healthy) was obtained from normal athymic mice.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Homyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the terms “treat.” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of glioma, delay or slowing of glioma, and amelioration or palliation of symptoms associated with glioma.

As used herein, the term “administering,” refers to the placement an agent as disclosed herein into a subject by a method or route which results in at least partial localization of the agents at a desired site.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. A subject that has a cancer or a tumor is a subject having objectively measurable cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs. As used herein, the term “invasive” refers to the ability to infiltrate and destroy surrounding tissue. Melanoma is an invasive form of skin tumor. As used herein, the term “carcinoma” refers to a cancer arising from epithelial cells. Examples of cancer include, but are not limited to, brain tumor, nerve sheath tumor, breast cancer, colon cancer, carcinoma, lung cancer, hepatoccllular cancer, gastric cancer, pancreatic cancer, cervical cancer, ovarian cancer, liver cancer, bladder cancer, cancer of the urinary tract, thyroid cancer, renal cancer, renal cell carcinoma, carcinoma, melanoma, head and neck cancer, brain cancer, and prostate cancer, including but not limited to androgen-dependent prostate cancer and androgen-independent prostate cancer. Examples of brain tumor include, but are not limited to, benign brain tumor, malignant brain tumor, primary brain tumor, secondary brain tumor, metastatic brain tumor, glioma, glioblastoma multiforme (GBM), medulloblastoma, ependymoma, astrocytoma, pilocytic astrocytoma, oligodendroglioma, brainstem glioma, optic nerve glioma, mixed glioma such as oligoastrocytoma. low-grade glioma, high-grade glioma, supratentorial glioma, infratentorial glioma, pontine glioma, meningioma, pituitary adenoma, and nerve sheath tumor.

“Conditions” and “disease conditions,” as used herein may include, but are in no way limited to any form of neurovascular diseases, any form of malignant neoplastic cell proliferative diseases, and abnormal angiogenesis (e.g., tumor angiogenesis, insufficient angiogenesis, or excessive angiogenesis). Examples of neurovascular diseases include but are not limited to stroke, brain trauma, AVM, brain aneurysms, carotid disease, cervical artery dissection, and vascular malformations. Examples of malignant neoplastic cell proliferative diseases include but are not limited to cancer and tumor. Examples of cancer and tumor include, but are not limited to, brain tumor, breast cancer, colon cancer, carcinoma, lung cancer, hepatocellular cancer, gastric cancer, pancreatic cancer, cervical cancer, ovarian cancer, liver cancer, bladder cancer, cancer of the urinary tract, thyroid cancer, renal cancer, renal cell carcinoma, carcinoma, melanoma, head and neck cancer, brain cancer, and prostate cancer, including but not limited to androgen-dependent prostate cancer and androgen-independent prostate cancer.

The term “sample” or “biological sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “sample” also includes untreated or pretreated (or pre-processed) biological samples. In some embodiments, a sample can comprise one or more cells from the subject. In some embodiments, a sample can be a tumor cell sample, e.g. the sample can comprise cancerous cells, cells from a tumor, and/or a tumor biopsy.

The term “functional” when used in conjunction with “equivalent”, “analog”, “derivative” or “variant” or “fragment” refers to an entity or molecule which possess a biological activity that is substantially similar to a biological activity of the entity or molecule of which it is an equivalent, analog, derivative, variant or fragment thereof.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., brain tumors) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

The term “statistically significant” or “significantly” refers to statistical evidence that there is a difference. It is defined as the probability of making a decision to reject the null hypothesis when the null hypothesis is actually true. The decision is often made using the p-value.

As used herein, “variants” can include, but are not limited to, those that include conservative amino acid mutations, SNP variants, splicing variants, degenerate variants, and biologically active portions of a gene. A “degenerate variant” as used herein refers to a variant that has a mutated nucleotide sequence, but still encodes the same polypeptide due to the redundancy of the genetic code. In accordance with the present invention, the Ago-2 protein may be modified, for example, to facilitate or improve identification, expression, isolation, storage and/or administration, so long as such modifications do not reduce Ago-2's function to unacceptable level. In various embodiments, a variant of the Ago-2 protein has at least 10%, 20%, 30%, 40%, 50%, 60%, 70%, 80%, 90%, or 95% of the function of a wild-type Ago-2 protein.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Cerebral arteriovenous malformation (AVM) is a vascular disease exhibiting abnormal blood vessel morphology and function. Current medical treatments for cerebrovascular disorders involve highly invasive procedures such as microsurgery, stereotactic radiosurgery and/or endovascular embolization. Moreover, difficult access to the brain region of interest (e.g. AVM nidus) can represent a significant risk to nearby eloquent cortical. subcortical, and neurovascular structures. Although these therapies are traditionally considered curative, AVM may recur, underlining the importance for the development of more efficient and safer therapies. The use of pharmaceutical drugs faces an important challenge which is the successful crossing of the blood-brain barrier (BBB), responsible for the low efficacy of drugs given systemically.

Exogenous application of miR-18a ameliorates the abnormal characteristics of AVM-derived brain endothelial cells (AVM-BEC) without the use of transfection reagents. In this application, we identify the mechanisms by which mir-18a is internalized by AVM-BEC, and explore the clinical application of a systemic miRNA carrier.

Primary cultures of AVM-BEC were isolated from surgical specimens and tested for endogenous miR-18a levels using qPCR. Conditioned media (CM) was derived from AVM-BEC cultures (AVM-BEC-CM). Ago-2 was detected using western blotting and immunostaining techniques. Secreted products (e.g., thrombospondin-1 (TSP-1)) were tested using ELISA. In the in vivo angiogenesis glioma model, animals were treated with miR-18a in combination with Ago-2. Plasma was obtained, and tested for TSP-1 and vascular endothelial growth factor (VEGF)-A.

AVM-BEC-CM significantly enhanced miR-18a internalization. Ago-2 was highly expressed in AVM-BEC; and siAgo-2 decreased miR-18a entry into brain-derived endothelial cells. Only brain-derived endothelial cells were responsive to the Ago-2/miR-18a complex and not other cell types tested. Brain endothelial cells treated with the Ago-2/miR-18a complex in vitro increased TSP-1 secretion. Using an in vivo angiogenesis model, the effects of the Ago-2/miR-18a complex caused a significant increase in TSP-1 and decrease in VEGF-A secretion in the plasma.

The functional effects of miR-18a on brain endothelial cells depend heavily on the presence of Ago-2. Without wishing to be bound by any particular theory, the requirement for this binary complex implies the existence/assembly of a putative cell membrane receptor for the ribonucleoprotein complex prior to internalization. Our studies found that brain endothelial cells are highly permissive to miRNA uptake compared to other endothelial cell types, such as the human microvascular endothelial cell line-1 (HMEC-1) and human umbilical vascular endothelial cells (HUVEC). Without wishing to be bound by any particular theory, this may be due to an intrinsic property of brain endothelial cells or is related to differences in ribonucleoprotein receptor density among the different endothelial cell types.

Taken together, Ago-2 facilitates miR-18a entry into AVM-brain endothelial cells in vitro and in vivo. Thus far, Ago-2 has been identified as an intracellular component of the RNA-induced silencing complex (RISC). However, we are the first to show that Ago-2 can be used as a specific miRNA carrier, particularly to the brain vasculature, with functional effects. This study demonstrates the clinical application of Ago-2 as a miRNA delivery platform for the treatment of brain vascular diseases.

The use of Ago-2 as a miRNA carrier and stabilizer overcomes all the limitations of systemic miRNA delivery by being able to carry functional miRNA specifically to the brain, thus traversing the BBB. The delivery of Ago-2/miRNA complex has significantly high target specificity and no apparent off-target effects while being minimally invasive (e.g., intravenous or intranasal administration).

This invention focuses on the therapeutic use of Argonaute-2 (Ago-2) as a carrier and stabilizer of miRNA for the treatment of brain vascular disorders. In our studies we show that Ago-2, a RNA-binding protein, forms a stable ribonucleoprotein complex with miRNA and can be used as an exogenous clinically-relevant agent. This Ago-2/miRNA complex is 1) internalized specifically by brain endothelial cells, 2) delivers functional miRNA, and 3) is clinically relevant based on in vivo activity. Ago-2 enhances miRNA stability allowing a more efficient miRNA internalization and consequent increased release of growth factors, e.g., thrombospondin-1 (TSP-1). TSP-1 is a key anti-angiogenic factor that antagonizes another pivotal molecule, vascular endothelial growth factor-A (VEGF-A). In vivo, the Ago-2/miRNA complex was administered systemically and effectively “normalized” the expression of key angiogenic factors to control plasma levels. Since the Ago-2/miRNA complex targets specifically the brain vasculature it has significant clinical relevance in the treatment of cerebrovascular diseases such as brain arteriovenous malformations (AVM), or diseases that involve active angiogenesis (i.e. stroke, angiogenesis in brain tumors). Furthermore, since Ago-2 is found in human circulation it is a biocompatible agent and therefore less likely to induce toxic side effects. Furthermore, Ago-2 can bind to several different miRNA; thus function will be related to activity of the miRNA. Hence, this carrier can be used for carrying a variety of miRNA sequences, and therefore target a variety of systems regulated by miRNA (e.g. growth factor expression, tumor suppression, neuronal development, cell differentiation and proliferation, immune system cell regulation). This opens new perspectives for the use of Ago-2 as a stable, safe and biocompatible miRNA carrier in different diseases. The current problem with miRNA-based therapy is inefficient delivery to the intended target tissue, off-target effects of miRNA and toxicity of the miRNA modulator (Noori-Daloii and Nejatizadeh; 2011). The delivery of miRNA with Ago-2 bypasses all these issues and can efficiently be administered through the intravenous or the intranasal route, as shown by our in vivo studies. For the reasons mentioned above, Ago-2/miRNA treatment is a safe and efficient therapeutic approach that can be used in the clinic.

MiRNA are small non-coding RNA that regulates protein expression by targeting messenger RNA for cleavage or translational repression. MiRNA-based therapy has great potential but faces several physiological obstacles. However, without wishing to be bound by any particular theory, it is believed that the use of Ago-2 as a miRNA carrier offers several advantages:

1) Ago-2 protects miRNA from intravascular degradation—intravenous naked delivery of miRNA often leads to degradation or renal clearance, meaning that the kidneys and other highly vascularized organs are preferred targets for this approach. Other research groups have tried chemical modification of these oligoribonucleotides for stabilization but they have low membrane penetration efficacy. Another alternative is the use of nanoparticle carriers; however, nanoparticles are often trapped by the reticuloendothelial system in the liver, lung and bone marrow, resulting in degradation by activated immune cells. Also, the physical and chemical properties of the nanoparticle surface can lead to hemolysis, thrombogenicity and complement activation, resulting in altered biodistribution and potential toxicity.

2) Ago-2 specifically and efficiently delivers miRNA to the endothelial cells of the brain vasculature—miRNA alone has low tissue penetrance and poor intracellular delivery, which can be overcome by using of transfection reagents e.g., lipofectamine (highly toxic in vivo), structural alterations of the miRNA (which offer low tissue penetrance), and nanoparticle/vesicle encapsulation. Many nanoparticles are internalized by endocytosis which can lead to miRNA degradation because lysosomes, which have an acidified (pH ˜4.5) contain nucleases.

3) Ago-2 complex formation does not require modification of miRNA thus function is maintained—chemical modification of miRNA such as 2′-O-methylation of the lead strand, intended to decrease intravascular degradation and immune system activation, lowers off-target effects without loss of activity but has poor internalization efficiency.

The growing number of miRNA sequences and their functions opens new perspectives of treatment for the use of a stable, safe and biocompatible miRNA carrier. Therefore our invention has strong therapy-based applications for the treatment of cerebrovascular disorders, stroke and brain tumors, which depend largely on the regulation of angiogenesis (formation of new blood vessels).

Methods of Delivering miRNA

In various embodiments, the present invention provides a method of delivering a miRNA to a cell. The method comprises or consists of: providing a miRNA and an Ago-2 or a variant thereof; and contacting the cell with the miRNA and the Ago-2 or the variant thereof, thereby delivering the mRNA to the cell. In some embodiments, the miRNA and the Ago-2 or the variant thereof are provided in one composition. In other embodiments, the miRNA and the Ago-2 or the variant thereof are provided in two separate compositions.

In various embodiments, the present invention provides a method of delivering a miRNA to a cell. The method comprises or consists of: providing a miRNA and an Ago-2 or a variant thereof; mixing the miRNA with the Ago-2 or the variant thereof; and contacting the cell with the mixture of the miRNA and the Ago-2 or the variant thereof, thereby delivering the mRNA to the cell. In various embodiments, the miRNA and the Ago-2 or the variant thereof form a ribonucleoprotein complex in the mixture.

In various embodiments, the present invention provides a method of delivering a miRNA to a cell. The method comprises or consists of: providing a composition comprising the miRNA and an Ago-2 or a variant thereof; and contacting the cell with the composition, thereby delivering the mRNA to the cell. In various embodiments, the miRNA and the Ago-2 or the variant thereof form a ribonucleoprotein complex in the composition.

In various embodiments, the cell is an endothelial cell or a brain endothelial cell. In some embodiments, the cell is in a sample or biological sample. In other embodiments, the cell is in a subject.

In various embodiments, the miRNA is miR-18a or miR-128a. In some embodiments, the miRNA is a miRNA suppressing angiogenesis (e.g., miR-92, miR-92a, miR-221/22). In other embodiments, the miRNA is a miRNA promoting angiogenesis (e.g., miR-296, miR-126, mir-210, miR-130).

In various embodiments, the Ago-2 can be a wild-type Ago-2 or recombinant Ago-2. In various embodiments, the variant of Ago-2 is a functional variant, equivalent, analog, derivative, or salt of Ago-2. In various embodiments, the Ago-2 or the variant thereof can be from any source, e.g., rat, mouse, guinea pig, dog, cat, rabbit, pig, cow, horse, goat, donkey or human.

### Treatment Methods

In various embodiments, the present invention provides a method of inhibiting angiogenesis in a subject. The method comprises or consists of: providing a miRNA and an Ago-2 or a variant thereof; and administering a therapeutically effective amount of the miRNA and the Ago-2 or the variant thereof to the subject, thereby inhibiting angiogenesis in the subject. In some embodiments, the miRNA and the Ago-2 or the variant thereof are provided in one composition. In other embodiments, the miRNA and the Ago-2 or the variant thereof are provided in two separate compositions. In various embodiments, the angiogenesis is angiogenesis in brain. In various embodiments, the angiogenesis is tumor angiogenesis. In various embodiments, the miRNA is a miRNA capable of inhibiting or suppressing angiogenesis. Non-limiting examples of miRNAs capable of inhibiting or suppressing angiogenesis include miR-92, miR-92a, and miR-221/22.

In various embodiments, the present invention provides a method of inhibiting angiogenesis in a subject. The method comprises or consists of: providing a miRNA and an Ago-2 or a variant thereof; mixing the miRNA with the Ago-2 or the variant thereof; and administering a therapeutically effective amount of the mixture to the subject. thereby inhibiting angiogenesis in the subject. In various embodiments, the miRNA and the Ago-2 or the variant thereof form a ribonucleoprotein complex in the mixture.

In various embodiments, the present invention provides a method of inhibiting angiogenesis in a subject. The method comprises or consists of: providing a composition comprising a miRNA and an Ago-2 or a variant thereof; and administering a therapeutically effective amount of the composition to the subject, thereby inhibiting angiogenesis in the subject. In various embodiments, the miRNA and the Ago-2 or the variant thereof form a ribonucleoprotein complex in the composition.

In various embodiments, the present invention provides a method of promoting angiogenesis in a subject. The method comprises or consists of: providing a miRNA and an Ago-2 or a variant thereof: and administering a therapeutically effective amount of the miRNA and the Ago-2 or the variant thereof to the subject, thereby promoting angiogenesis in the subject. In some embodiments, the miRNA and the Ago-2 or the variant thereof are provided in one composition. In other embodiments, the miRNA and the Ago-2 or the variant thereof are provided in two separate compositions. In various embodiments, the miRNA is a miRNA capable of promoting angiogenesis. Non-limiting examples of miRNAs capable of promoting angiogenesis include miR-296, miR-126, mir-210, miR-130.

In various embodiments, the present invention provides a method of promoting angiogenesis in a subject. The method comprises or consists of: providing a miRNA and an Ago-2 or a variant thereof; mixing the miRNA with the Ago-2 or the variant thereof; and administering a therapeutically effective amount of the mixture to the subject, thereby promoting angiogenesis in the subject. In various embodiments, the miRNA and the Ago-2 or the variant thereof form a ribonucleoprotein complex in the mixture.

In various embodiments, the present invention provides a method of promoting angiogenesis in a subject. The method comprises or consists of: providing a composition comprising a miRNA and an Ago-2 or a variant thereof; and administering a therapeutically effective amount of the composition to the subject, thereby promoting angiogenesis in the subject. In various embodiments, the miRNA and the Ago-2 or the variant thereof form a ribonucleoprotein complex in the composition.

In various embodiments, the present invention provides a method of treating, preventing, reducing the likelihood of having, reducing the severity of and/or slowing the progression of a condition in a subject. The method comprises or consists of: providing a miRNA and an Ago-2 or a variant thereof; and administering a therapeutically effective amount of the miRNA and the Ago-2 or the variant thereof to the subject, thereby treating, preventing, reducing the likelihood of having, reducing the severity of and/or slowing the progression of the condition in the subject.

In some embodiments, the miRNA and the Ago-2 or the variant thereof are provided in one composition. In other embodiments, the miRNA and the Ago-2 or the variant thereof are provided in two separate compositions. In various embodiments, the condition is a neurovascular disease. In various embodiments, the condition is cerebral arteriovenous malformations (AVM) or stroke. In various embodiments, the condition is a tumor. In various embodiments, the condition is brain tumor, glioma. glioblastoma, andior glioblastoma multiforme (GBM).

In various embodiments, the present invention provides a method of treating, preventing, reducing the likelihood of having, reducing the severity of and/or slowing the progression of a condition in a subject. The method comprises or consists of: providing a miRNA and an Ago-2 or a variant thereof; mixing the miRNA and the Ago-2 or the variant thereof and administering a therapeutically effective amount of the mixture to the subject, thereby treating, preventing, reducing the likelihood of having, reducing the severity of and/or slowing the progression of the condition in the subject. In various embodiments, the miRNA and the Ago-2 or the variant thereof form a ribonucleoprotein complex in the mixture.

In various embodiments, the present invention provides a method of treating, preventing, reducing the likelihood of having, reducing the severity of and/or slowing the progression of a condition in a subject. The method comprises or consists of: providing a composition comprising a miRNA and an Ago-2 or a variant thereof; and administering a therapeutically effective amount of the composition to the subject, thereby treating, preventing, reducing the likelihood of having, reducing the severity of and/or slowing the progression of the condition in the subject. In various embodiments, the miRNA and the Ago-2 or the variant thereof form a ribonucleoprotein complex in the composition.

In various embodiments, the subject is a human. In various embodiments, the subject is a mammalian subject including but not limited to human, monkey, ape, dog, cat, cow, horse, goat, pig, rabbit, mouse and rat.

In various embodiments, the miRNA is miR-18a or miR-128a. In some embodiments, the miRNA is a miRNA suppressing angiogenesis (e.g. miR-92, miR-92a, miR-221/22).

In various embodiments, the Ago-2 can be a wild-type Ago-2 or recombinant Ago-2. In various embodiments, the variant of Ago-2 is a functional variant, equivalent, analog, derivative, or salt of Ago-2. In various embodiments, the Ago-2 or the variant thereof can be from any source, e.g., rat, mouse, guinea pig, dog, cat, rabbit, pig, cow, horse, goat, donkey or human.

In various embodiments, the miRNA is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L. In certain embodiments, the miRNA is administered to a human.

In various embodiments, the Ago-2 or the variant thereof is administered at about 0.001 to 0.01, 0.01 to 0.1.0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L. In certain embodiments, the Ago-2 or the variant thereof is administered to a human.

Typical dosages of an effective amount of the miRNA or the Ago-2 or the variant thereof can be in the ranges recommended by the manufacturer where known therapeutic compounds are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models. In various embodiments, the miRNA and the Ago-2 or the variant thereof are administered once a day (SID/QD), twice a day (BID), three times a day (TID), four times a day (QID), or more, so as to administer an effective amount of the miRNA and the Ago-2 or the variant thereof to the subject, where the effective amount is any one or more of the doses described herein.

In accordance with the invention, the mixture is administered using the appropriate modes of administration, for instance, the modes of administration recommended by the manufacturer for each of the miRNA and the Ago-2 or the variant thereof. In accordance with the invention, various routes may be utilized to administer the mixture of the claimed methods, including but not limited to intratumoral, intracranial, intravcntricular, intrathccal, epidural, intradural, aerosol, nasal, oral, transmucosal. transdermal, parenteral, implantable pump, continuous infusion, topical application, capsules and/or injections. In various embodiments, the mixture is administered intratumorally, intracranially, intraventricularly, intrathecally, epidurally, intradurally, intravascularly, intravenously, intraarterially, intramuscularly, subcutaneously, intraperitoneally, intranasally, or orally. In further embodiments, the mixture is administered with food or without food.

In various embodiments, the mixture is administered once, twice, three or more times. In various embodiments, the mixture is administered 1-3 times per day, 1-7 times per week, or 1-9 times per month. In various embodiments, the mixture is administered for about 1-10 days, 10-20 days, 20-30 days, 30-40 days, 40-50 days. 50-60 days. 60-70 days. 70-80 days, 80-90 days, 90-100 days, 1-6 months, 6-12 months, or 1-5 years.

Various method described herein can further comprise providing and administering a therapeutically effective amount of an anti-angiogenic drug to the subject. In various embodiments, the mixture and the anti-angiogenic drug are administered concurrently or sequentially. In various embodiments, the mixture is administered before, during or after administering the anti-angiogenic drug. As a non-limiting example, the mixture may be administered, for example, daily at the aforementioned dosages, and the anti-angiogenic drug may be administered, for example, daily, weekly, biweekly, every fortnight and/or monthly at the aforementioned dosages. As another non-limiting example, the mixture may be administered, for example, daily, weekly, biweekly, every fortnight and/or monthly, at the aforementioned dosages, and the anti-angiogenic drug may be administered, for example, daily at the aforementioned dosages. Further, each of the mixture and the anti-angiogenic drug may be administered daily, weekly, biweekly, every fortnight and/or monthly, wherein the mixture is administered at the aforementioned dosages on a day different than the day on which the anti-angiogenic drug is administered at the aforementioned dosages. In some embodiments, the mixture and the anti-angiogenic drug are in one composition or separate compositions.

In accordance with the present invention, examples of anti-angiogenic drugs include but are not limited to Genentech/Roche (Bevacizumab/Avastin®), Bayer and Onyx Pharmaceuticals (sorafenib/Nexavar®), Pfizer (sutinib/Sutent®), GlaxoSmithKline (pazopanib/Votrient®), Novartis (everolimus/Affinitor®), Celgene (poinalidomide/Pomalyst®) and Ipsen and Active Biotech (tasquinimod/ABR-215050, CID 54682876).

Various method described herein can further comprise providing and administering a therapeutically effective amount of a chemotherapeutic agent to the subject. In accordance with the invention, the mixture and the chemotherapeutic agent are administered concurrently or sequentially. Still in accordance with the invention, the mixture is administered before, during or after administering the chemotherapeutic agent. As a non-limiting example, the mixture may be administered, for example, daily at the aforementioned dosages, and the chemotherapeutic agent may be administered, for example, daily, weekly, biweekly, every fortnight and/or monthly at the aforementioned dosages. As another non-limiting example, the mixture may be administered, for example, daily, weekly, biweekly, every fortnight and/or monthly, at the aforementioned dosages, and the chemotherapeutic agent may be administered, for example, daily at the aforementioned dosages. Further, each of the mixture and the chemotherapeutic agent may be administered daily, weekly, biweekly, every fortnight and/or monthly, wherein the mixture is administered at the aforementioned dosages on a day different than the day on which the chemotherapeutic agent is administered at the aforementioned dosages. In some embodiments, the mixture and the chemotherapeutic agent are in one composition or separate compositions.

In accordance with the present invention, examples of the chemotherapeutic agent include but are not limited to Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine. Vorinostat, Romidepsin, 5-fluorouracil (5-FU), 6-mercaptopurine (6-MP), Cladribine, Clofarabine, Floxuridine, Fludarabine, Pentostatin, Mitomnycin, ixabepilone, Estramustine, prednisone, methylprednisolone, dexamethasone or a combination thereof.

### Pharmaceutical Compositions

In some embodiments, the miRNA and the Ago-2 or the variant thereof are provided in one composition. In other embodiments, the miRNA and the Ago-2 or the variant thereof are provided in separate compositions. In various embodiments, the present invention provides a composition that comprises or consists of a miRNA and an Ago-2 or a variant thereof. In various embodiments, the present invention provides a composition that comprises or consists of a ribonucleoprotein complex of a miRNA and an Ago-2 or a variant thereof. In accordance with the present invention, various compositions described herein may be used for delivering miRNA to a cell, inhibiting angiogenesis, promoting angiogenesis, and/or treating, preventing, reducing the likelihood of having, reducing the severity of and/or slowing the progression of a condition in a subject.

In various embodiments. the angiogenesis is angiogenesis in brain. In various embodiments, the angiogenesis is tumor angiogenesis. In various embodiments, the condition is a neurovascular disease. In various embodiments, the condition is cerebral arteriovenous malformations (AVM) or stroke. In various embodiments, the condition is a tumor. In various embodiments, the condition is brain tumor, glioma, glioblastoma, and/or glioblastoma multiforme (GBM). In certain embodiments, the composition is administered to a human.

In various embodiments, the miRNA is miR-18a or miR-128a. In some embodiments, the miRNA is a miRNA suppressing angiogenesis (e.g. miR-92, miR-92a, miR-221/22). In various embodiments, the composition comprises about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L miRNA.

In various embodiments, the Ago-2 can be a wild-type Ago-2 or recombinant Ago-2. In various embodiments, the variant of Ago-2 is a functional variant, equivalent, analog, derivative, or salt of Ago-2. In various embodiments, the Ago-2 or the variant thereof can be from any source, e.g., rat, mouse, guinea pig, dog, cat, rabbit, pig, cow, horse, goat, donkey or human. In various embodiments, the composition comprises about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L Ago-2 or a variant thereof.

In various embodiments, the composition further comprises an anti-angiogenic drug. In various embodiments, the composition further comprises a chemotherapeutic agent.

In accordance with the invention, the miRNA and the Ago-2 or the variant thereof useful in the treatment of disease in mammals will often be prepared substantially free of naturally-occurring immunoglobulins or other biological molecules. Preferred miRNAs and/or Ago-2s or variants thereof will also exhibit minimal toxicity when administered to a mammal.

In various embodiments, the pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers. stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art. In various embodiments, the composition is formulated for intratumoral, intracranial, intraventricular, intrathecal. epidural, intradural, intravascular, intravenous, intraarterial, intramuscular, subcutaneous, intraperitoneal. intranasal, or oral administration.

In various embodiments, the composition is administered 1-3 times per day, 1-7 times per week, or 1-9 times per month. In various embodiments, the composition is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In various embodiments, the composition is administered once a day (SID/QD), twice a day (BID), three times a day (TID), four times a day (QID), or more, so as to administer an effective amount of the miRNA and the Ago-2 or the variant thereof to the subject, where the effective amount is any one or more of the doses described herein.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ. or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky el al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

### Kits of the Invention

In various embodiments, the present invention provides a kit for delivering a miRNA to a cell. The kit comprises or consists of: a quantity of a miRNA, a quantity of an Ago-2 or a variant thereof; and instructions for using the Ago-2 or the variant thereof to deliver the miRNA.

In various embodiments, the present invention provides a kit for inhibiting angiogenesis in a subject. The kit comprises or consists of: a quantity of a miRNA; a quantity of an Ago-2 or a variant thereof; and instructions for using the miRNA and the Ago-2 or the variant thereof to inhibit angiogenesis in the subject.

In various embodiments, the present invention provides a kit for treating, preventing, reducing the severity of and/or slowing the progression of a condition in a subject. The kit comprises or consists of: a quantity of a miRNA; a quantity of an Ago-2 or a variant thereof; and instructions for using the miRNA and the Ago-2 or the variant thereof to treat, prevent, reduce the likelihood of having, reduce the severity of and/or slow the progression of the condition in the subject.

In various embodiments, the kits described herein can further comprise an anti-angiogenic drug and/or chemotherapeutic agent, and instructions for using the anti-angiogenic drug and/or chemotherapeutic agent to inhibit angiogenesis and/or to treat, prevent, reduce the likelihood of having, reduce the severity of and/or slow the progression of the condition in the subject.

The kit is an assemblage of materials or components, including at least one of the inventive compositions. Thus, in some embodiments the kit contains a composition including a drug delivery molecule complexed with a therapeutic agent, as described above.

The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use can be included in the kit. “Instructions for use” typically include a tangible expression describing the technique to be employed in using the components of the kit to affect a desired outcome. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components.

### EXAMPLES

The following examples are offered for illustrative purposes only, and are not intended to limit the scope of the present invention in any way.

### Example 1: Methods and Materials

### Endothelial Cell Isolation and Culture

Human surgical specimens were obtained in accordance with guidelines set forth by the Institutional Review Board (HS-04B053), at Keck School of Medicine, University of Southern California, and in accordance with Animal Research: Reporting In Vivo Experiments (ARRIVE) guidelines. AVM-BEC were obtained from brain tissues of 6 patients who underwent AVM resection; control BEC were isolated from structurally normal cortex of 4 epileptic patients as described previously (Stapleton et al., Thrombospondin-1 modulates the angiogenic phenotype of human cerebral arteriovenous malformation endothelial cells. Neurosurgery. 2011:68:1342-1353). Primary cell cultures were used only until passage 5. Human umbilical vein endothelial cells (HUVEC) and human dermal microvascular endothelial cell line (HMEC) were maintained in RPMI media containing fetal calf serum (FCS).

Cells were treated with miR-18a (40 nmol/L), siAgo-2 (30-75 nmol/L), lipofectamine (2 μg/ml) (Life Technologies, Carlsbad, Calif.). A scrambled miRNA sequence (50-75 nmol/L) and siGFP (40 nM; Life Technologies) were used as negative controls. siGFP was chosen as a negative control since the miR-18a mimic is a double-stranded RNA and these cells do not express green fluorescent protein (GFP). Cells were treated with varying concentrations of human recombinant Ago-2 (0.01-80 nmol/L; Abcam, San Francisco, Calif.) for 30 minutes to determine its activity as a miRNA carrier. For inhibition of exosome release GW4869 (5-50 μmol/L; Sigma, St. Louis, Mo.) was used for 24 hours. All experiments were performed under arterial shear flow (12 dyn/cm²), as reported previously (Ferreira et al., Microrna-18a improves human cerebral arteriovenous malformation endothelial cell function. Stroke. 2014; 45:293-297).

miRNA Extraction and Detection

After thoroughly washing, cells were lysed with lysis butffer (Ambion®, ThermoScientific, Pittsburgh, Pa.) and miRNA was extracted using mirVana™ miRNA Isolation Kit (Ambion®) and transcribed using TaqMan MicroRNA Reverse Transcription Kit (Applied Biosystems, Foster City, Calif.). Gene expression was analyzed by qPCR using TaqMan Universal Master Mix II (Life Technologies), TaqMan® Assay for miR-18a and TaqMan® Control miRNA Assay for RNU44 (Applied Biosystems), per manufacturer's instructions, using a Stratagene Mx3000P Bioanalyzer (Agilent Technologies, Westlake Village, Calif.). PCR products were normalized to RNU44, an endogenous small-nucleolar RNA with stable expression within all cell types tested. For 4° C. experiments, cells were maintained at 4° C. for 10 minutes before treatments, and maintained at that temperature for an additional 30 minutes before cell lysis.

### Conditioned Media (CM) Experiments

Cells were seeded at a density of 2×10⁴ cells/well in complete media into 24 well tray plates for 24 hours and allowed to become 70-80% confluent; media was then removed and cells incubated in FCS-free RPMI medium supplemented with penicillin and streptomycin, for 24 hours. CM was collected and centrifuged at 2.000 rpm at 4° C. for 10 minutes. CM was then used without further dilution for subsequent experiments unless stated otherwise.

Quantitative Real-Time Polymerase Chain Reaction (qPCR)

Gene expression was confirmed by qPCR using iQ™ SYBR® Green Supermix (BioRad, Hercules, Calif.) according to the manufacturer's instructions using a Stratagene Mx3000P Bioanalyzer (Agilent Technologies, Westlake Village, Calif.). PCR products were normalized to 18S ribosomal ribonucleic acid (18S rRNA). The following forward and reverse primer sequences were used, respectively: NPM-1, 5′-AGCACTTAGTAGCTGTGGAG-3′ (SEQ ID NO:1); 5′-CTGTGGAACCTTGCTACCACC-3′ (SEQ ID NO:2); NCL, 5′-GGTGGTTTCCCAACAAA-3′ (SEQ ID NO:3); 5′-GCCAGGTGTGGTAACTGCT-3′ (SEQ ID NO:4); Ago-2, 5′-GTTTGACGGCAGGAAGAATCT-3′ (SEQ ID NO:5); 5′-AGGACACCCACTTGATGGACA-3′ (SEQ ID NO:6); 18S rRNA, 5′-CGGCTACCACATCCAAGGAA-3′ (SEQ ID NO:7); 5′-GCTGGAATTACCGCGGCT-3′ (SEQ ID NO:8).

### Western Blot

Total protein was extracted and quantified using the Bicinchoninic Acid Protein Assay Kit (Thermo Fisher Scientific). Equal amounts of protein were separated by sodium dodecyl sulfate polyacrylamide gel electrophoresis and transferred to 0.45-μm polyvinylidene fluoride microporous membranes. Membranes were blocked with Sea Block (Thermo Fisher Scientific), probed with anti-Ago-2 (1:1,000) (Cell Signaling Technology Inc., Beverly, Mass.) or anti-actin (1:1,500) (Santa Cruz Biotechnology) antibodies, and incubated with the appropriate fluorescent secondary anti-rabbit antibody (1:15,000) (Thermo Fisher Scientific). Protein bands were detected by Odyssey infrared imaging (LI-COR Biosciences, Lincoln, Nebr.) and densitometric studies were performed using NIH free software ImageJ. Actin levels were measured for internal standardization.

### Immunocytochemistry

Cells were fixed with 4% paraformaldehyde (PFA) and then washed with PBS. Nonspecific binding was prevented using Sea Block blocking solution (Thermo Fisher Scientific, Rockford, Ill.). Cells were kept overnight at 4° C. in a primary antibody solution and incubated for 1 hour at RT with the corresponding secondary antibody. Antibodies were used as listed: rabbit anti-Ago-2 (1:100) (Cell Signaling Technology Inc.); rabbit anti-CD8 (1:50) (Santa Cruz Biotechnology Inc., Dallas, Tex.); Alexa Fluor 594 goat anti-rabbit (1:200) (Molecular Probes, Oregon, USA). For nuclear labeling, cell preparations were stained with Hoechst-33342 (2 μg/ml) (Sigma) and mounted in Dako Fluorescence Mounting Medium (Dako North America Inc., Carpinteria, Calif.). Fluorescent images were acquired using an LSM 510 confocal microscope with a 40× objective (Carl Zeiss Inc., Dublin, Calif.).

### Immunoprecipitation

Rabbit anti-Ago2 (Abcam Inc., Cambridge, Boston), or rabbit normal IgG (Santa Cruz Biotechnology) antibodies were pre-incubated with Magna Bind goat anti-rabbit IgG Magnetic Bead slurry (Thermo Scientific) and used for immunoprecipitation overnight of the following preparations: Ago-2 alone (0.4 nmol/L; Abcam), miR-18a alone (40 nmol/L), or miR-18a (40 nmol/L) plus Ago-2 (40 nmol/L). One half of the sample was analyzed by SDS/PAGE and immunoblotting. The other half was processed for miRNA isolation (Ambion®).

### Enzyme-Linked Immunosorbent Assay (ELISA)

Cell supernatants were collected, filtered through a 0.2-μm cellulose acetate membrane (VWR International, West Chester, Pa.) and analyzed for TSP-1 (R&D Systems, Minneapolis, Minn.) using commercially available ELISA kits per manufacturer's instructions. Remaining cells were lysed and total amount of protein was determined for normalization.

For in vivo experiments, mouse blood samples (800 μl) were collected in heparin, plasma obtained, and further analyzed for TSP-1 and VEGF-A using commercially available ELISA kits (R&D Systems).

### In Vivo Experiments

Animal protocols were approved by Institutional Animal Care and Use Committee (IACUC) of the University of Southern California. Renilla luciferase-labeled U251 glioma cells (2×10⁵ cells) were implanted intracranially into 4-5 week old male athymic/nude mice (Harlan Laboratories, Inc., Placentia, Calif.), as previously described (Virrey et al., Glioma-associated endothelial cells are chemoresistant to temozolomide. J. Neurooncol. 2009; 95:13-22). Animals were anesthetized with ketamine (100 mg/kg)/xylazine (10 mg/kg) administered intraperitoneally prior to implantation and treated subcutaneously with buprenorphine (0.06 mg/kg) daily for 2 days post-implantation. Imnaging was performed after 3 days to group mice with similar imaging intensities, as evaluated by region of interest (ROI) values. The following treatment groups were established: vehicle (PBS) (n=5); miR-18a (40 nmol/L) in combination with Ago-2 (0.4 nmol/L) (n=5); miR-18a alone (40 nmol/L) (n=5); Ago-2 alone (0.4 nmol/L) (n=5). Treatments were administered intravenously through the tail vein every 48 hours (for a total of 3 treatment sessions). Mice were injected with 1 mg/kg ViviRen™ In Vivo Renilla Luciferase Substrate (Promega Madison, Wis.) intravenously and imaged 3 days post-injection to determine baseline tumor growth and 6 days after treatments using the IVIS 200 optical imaging system (Caliper Life Sciences, Hopkinton, Mass.); images were analyzed using LIVING IMAGE software (Caliper Life Sciences).

### Statistical Analysis

Statistical analysis was performed using GraphPad Prism 5.0 (GraphPad Software, San Diego, Calif.). Statistical significance was considered relevant for p values <0.05 using one-way analysis of variance followed by Bonferroni or Dunnett post hoc test. Data are presented as mean±standard error of the mean (SEM). Every experimental condition was tested in three sets of independent experiments unless stated otherwise, and performed in duplicates. For every immunocytochemistry analysis, five independent microscopy fields were acquired per coverslip, with a 40× objective (approximately 40 cells per field).

### Example 2: AVM-BEC-Conditioned Media Potentiates miR-18a Entry

Our experimental model is based on the use of AVM-derived brain endothelial cells (AVM-BEC) isolated from brain tissue of six patients who underwent microsurgical AVM resection. We found no correlation between any of the clinical parameters listed and the results obtained.

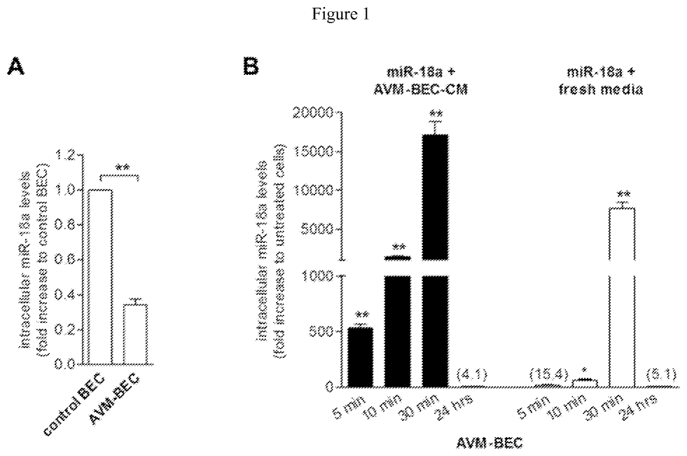

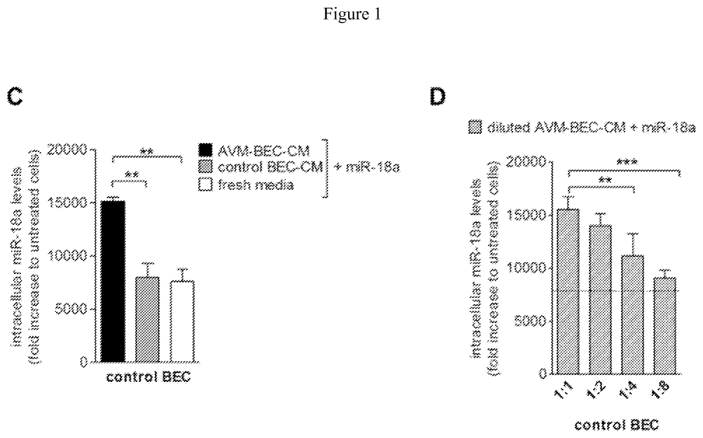

It is shown that miR-18a (40 nmol/L) can be internalized by AVM-BEC without transfection agents. resulting in functional changes including a significant increase in thrombospondin-1 (TSP-1) release, and decrease in vascular endothelial growth factor-A (VEGF-A). Based on these studies, we proceeded to explore the mechanism of entry of miR-18a into AVM-BEC. We first analyzed the endogenous expression of miR-18a in AVM-BEC and control BEC using qPCR; AVM-BEC endogenously express significantly less miR-18a as compared to control BEC (AVM-BEC=0.34±0.03; n=3; p<0.01) (FIG. 1A). When cells were treated exogenously with miR-18a (40 nmol/L) for increasing periods of incubation (5 minutes to 24 hours) in the presence of AVM-BEC-conditioned media (AVM-BEC-CM) (black bars) or fresh media (white bars), we observed that uptake of miR-18a by AVM-BEC was enhanced, particularly with AVM-BEC-CM after 30 minutes of incubation (FIG. 1B). AVM-BEC-CM contained no FCS, to avoid contaminating nucleic acids, proteins and transporting microvesicles. AVM-BEC-CM treatment resulted in higher levels of detected intracellular miR-18a as compared to fresh media for each time point (AVM-BEC-CM_{5 min}=533.2±33.4; fresh_{5 min}=15.4±2.5; AVM-BEC-CM_{10 min}=1465.6±43.0; fresh_{10 min}=62.4±7.9: AVM-BEC-CM_{30 min}=17136.0±1697.0; fresh_{30 min}=7668.7=783.4; n=3-4; *p<0.05, **p<0.01). Incubation times longer than 30 minutes proved to be less effective; there were no significant differences observed at 24 hours (AVM-BEC-CM_{24 hrs}=4.1±0.7; Fresh_{24 hrs}=5.1±0.6; n=3) (FIG. 1B). Treatment with scramble miRNA or siGFP (40 nmol/L) in the presence of AVM-BEC-CM, BEC-CM or fresh media did no change intracellular miR-18a levels.

These data clearly show that AVM-BREC-CM enhanced entry of exogenous miR-18a. To assess whether AVM-BEC-CM affected miR-18a entry in control BEC, these cells were subjected to miR-18a treatment in the presence of AVM-BEC-CM (black bar), BEC-CM (gray bar) or fresh media (white bar), for 30 minutes (FIG. 1C). The results show that AVM-BEC-CM potentiated miR-18a entry in control BEC, compared to BEC-CM and fresh media (AVM-BEC-CM=15106.01419.2; BEC-CM=8012.111288.2; fresh=7606.011137.1; n=3; **p<0.01) (FIG. 1C). Diluted AVM-BEC-CM (1:2, 1:4 and 1:8) was used to determine if a soluble agent, in limiting amount, was promoting miRNA entry in miR-18a-treated cells (FIG. 1D). Increasingly diluted AVM-BEC-CM led to proportional decline of miR-18 internalization (1:1=15521.3±1216.0; 1:2=13951.4±1197.1; 1:4=11124.9±2112.3; 1:8=9097.8±663.5; n=3; p<0.01, p<0.001) (FIG. 1D). Thus a soluble agent secreted by AVM-BEC in AVM-BEC-CM is likely responsible for enhanced uptake of miR-18a.

### Example 3: AVM-BEC Express RNA-Binding Protein Ago-2

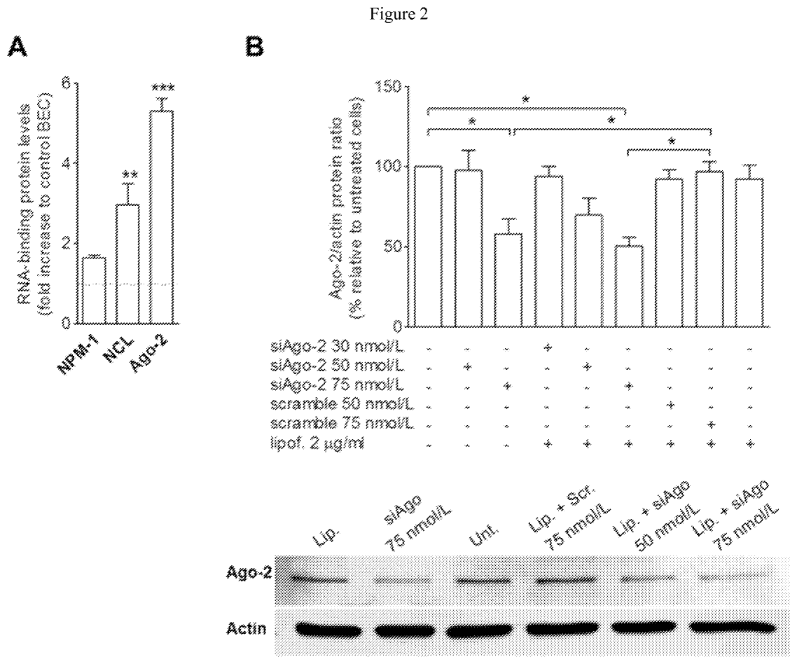

Without wishing to be bound by any particular theory, it is suggested that the main mechanism for the entry of extracellular RNA into cells occurs through the formation of ribonucleoprotein complexes. Therefore, we screened AVM-BEC as compared to control BEC for RNA-binding protein expression. AVM-BEC significantly express more nucleolin (NCL) and argonaute-2 (Ago-2) as compared to control BEC, while nucleophosmin 1 (NPM-1) was not significantly different (NPM-1=1.6±0.1; NCL=2.9±0.5; Ago-2=5.3±0.3; n=3; **p<0.01, ***p<0.001) (FIG. 2A). Given its high expression, we selected Ago-2 as a target for downregulation, and treated AVM-BEC with siAgo-2 (30-75 nmol/L) to determine the role of this RNA-binding protein in miR-18a delivery (FIG. 2B). The results of this experiment showed that treatment with siAgo-2 (75 nmol/L) alone, or siAgo-2 (75 nmol/L) with lipofectamine had similar and significant effects in reducing Ago-2 expression (siAgo-2=58.2±9.2; lipof.+siAgo-2=50.6±5.3; n=3; p<0.05). We tested the internalization of another miRNA mimic, miR-128a, and found internalization, but to a lesser extent than miR-18a (AVM-BEC=5.9±0.1; n=3; *p<0.05), which indicates that AVM-BEC can efficiently internalize universal exogenous miRNA without transfection reagents. These data showed that AVM-BEC-CM contains a delivery agent that works as effectively as lipofectamine (FIG. 2B). It was not possible to determine the amount of Ago-2 protein in AVM-BEC-CM because of the limited amount of conditioned media obtainable from these primary endothelial cell cultures. Nevertheless, siAgo-2 was effective in reducing intracellular Ago-2 levels in AVM-BEC (FIG. 2B). In subsequent experiments siAgo-2 was used in the presence of lipofectamine to induce the maximal decrease of Ago-2 levels.

### Example 4: Silencing Ago-2 Compromises the Entry of Exogenous miR-18a into Brain Endothelial Cells

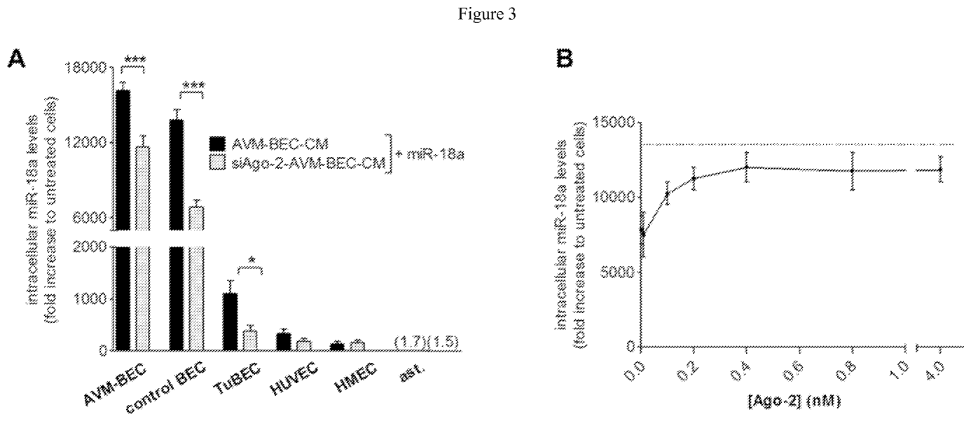

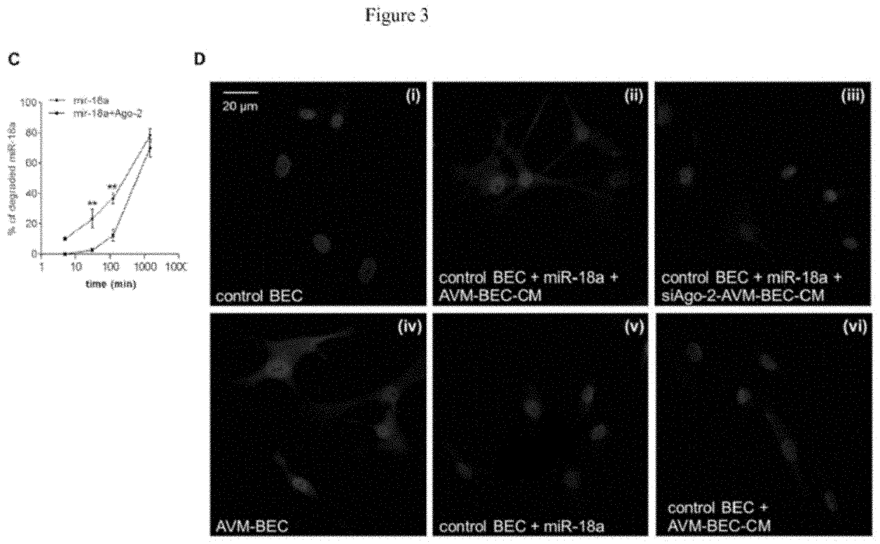

To determine the effects of decreased Ago-2 secreted levels, AVM-BEC were treated with siAgo-2 or scrambled siRNA; siAgo-2-AVM-BEC-CM was then collected and tested in the presence of miR-18a (FIG. 3A). AVM-BEC-CM was more effective than siAgo2-AVM-BEC in raising intracellular miR-18a levels after exogenous treatment; thus Ago-2 is important in transporting miR-18a. Primary endothelial cell cultures of brain origin such as AVM-BEC, control BEC and glioma-derived brain endothelial cells (TuBEC) showed the most significant miR-18a internalization with AVM-BEC-CM and the most significant reduction of internalized miR-18a in the presence of siAgo-2-AVM-BEC-CM (AVM-BEC_{AVM-BEC-CM}=16167.7±600.9; AVM-BEC_{siAgo2-AVM-BEC-CM}=11633.2±876.2; control BEC_{AVM-BEC-CM}=13815.7±823.4; control BEC_{siAgo-2-AVM-BEC-CM}=6856.2±545.0; TuBEC_{AVM-BEC-CM}=1104.1±249.1; TuBEC_{siAgo-2-AVM-BEC}=383.6±104.8; n=3; p<0.001: p<0.05). Interestingly, astrocytes did not exhibit increased intracellular miR-18a levels with AVM-BEC-CM and, accordingly, Ago-2 silencing had no effect on uptake (ast._{AVMS-BEC-CM}=1.7±0.1; ast._{siAgo2-AVM-BEC-CM}=1.5±0.1; n=2). There were also no significant differences in miRNA uptake with AVM-BEC-CM or siAgo-2-AVM-BEC-CM in human umbilical vein endothelial cells (HUVEC) or human dermal microvascular endothelial cell line (HMEC), (HUVEC_{AVM-BEC-CM}=367.7±80.9; HUVEC_{siAgo-2-AVM-BEC-CM}=180.3±61.4; HMEC_{AVM-BEC-CM}=134.3±54.6; HMEC_{siAgo-2-AVM-BEC-CM}=163.7±41.3; n=3) (FIG. 3A). These results correlated reduced Ago-2 levels (as observed in siAgo-2-AVM-BEC-CM) with decreased miR-18a uptake into brain endothelial cells. To further validate the role of Ago-2 as a miRNA carrier, control BEC were treated with miR-18a (40 nmol/L) and Ago-2 (0.01-0.8 nmol/L) (FIG. 3B). Increasing concentrations of exogenously applied Ago-2 (up to 0.4 nmol/L) led to increased detection of miR-18 intracellular levels (n=3). Additionally, miR-18a in combination with Ago-2 resulted in significantly lower miRNA degradation levels than miR-18a alone, particularly after and 120 minutes (mir-18a_{30 min}=23.3±6.0; mir-18a_{120 min}=36.7±3.3; mir-18a+Ago-2_{30 min}=2.7±1.2; mir-18a+Ago-2_{120 min}=12.3±3.9; n=3; p<0.01) (FIG. 3C). Without wishing to be bound by any particular theory, these data show that one mechanism by which Ago-2 increases miR-18a intracellular levels is that Ago-2 protects the miRNA from degradation.

Immunocytochemistry analysis of AVM-BEC and control BEC stained for Ago-2 showed that basal Ago-2 expression is higher in AVM-BEC than control BEC (FIG. 3D; (i) vs. (iv)). However, control BEC treated with miR-18a in the presence of AVM-BEC-CM showed increased intracellular Ago-2 labeling supporting the previous concept that Ago-2 acts as a carrier for miR-18a (FIG. 3D; (ii)). Conversely, control BEC treated with miR-18a in the presence of siAgo-2-AVM-BEC-CM did not show a significant signal (FIG. 3D; (iii)). AVM-BEC-CM alone, i.e. without added miR-18a, did not increase intracellular Ago-2, emphasizing the importance of having both Ago-2 and miR-18a available in the media to enhance internalization (FIG. 3D; (vi)).

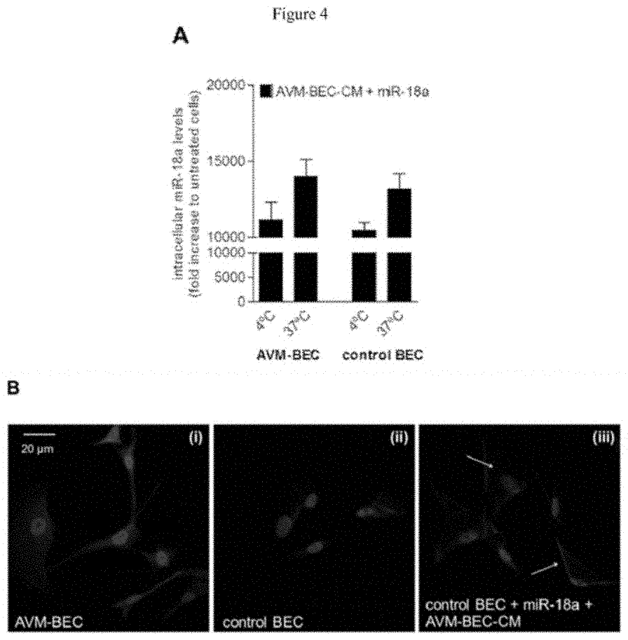

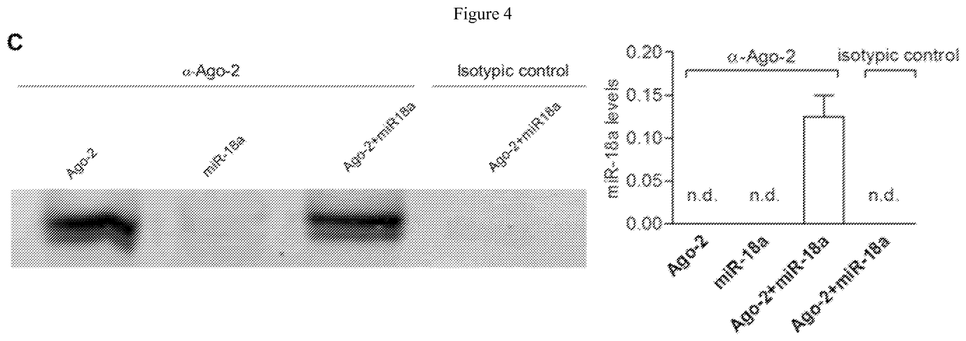

In an effort to clarify whether active transport was responsible for entry. miR-18a intracellular levels were quantified in recipient cells in the presence of AVM-BEC-CM and miR-18a treatment (40 nmol/L) at 4° C. and compared to 37° C. (FIG. 4A). AVM-BEC and control BEC subjected to cold temperature showed decreased miR-18a uptake, albeit not significantly (4° C._{AVM-BEC}=1167.7±1062.3; 37° C._{AVM-BEC}=14080.1±1155.4; 4° C._{control BEC}=10467.1±517.5; 37° C._{control BEC}=13167.1±1014.8: n=3) suggesting that miRNA delivery could involve passive transport. However, at low temperatures Ago-2 may adhere to the cell surface, resulting in a miR-18a and Ago-2 complex adsorbed onto the outer surface of the membrane; this lead to an overestimate of intracellular miR-18a levels when cell lysates were evaluated. Accordingly, Confocal microscopy studies revealed that at 4° C. in the presence of AVM-BEC-CM and miR-18a control BEC were positive for Ago-2 staining on the cell surface despite rigorous washing (FIG. 4B). Therefore, the data showing high levels of miR-18a at are likely the result of Ago-2 adsorption onto the cell surface, rather than passive cellular uptake.

To determine whether Ago-2 formed a ribonucleoprotein complex with miR-18a, we performed Ago-2 immunoprecipitation, followed by immunoblotting for Ago-2 and qPCR analysis for miRNA detection (FIG. 4C). Ago-2 was only detected in the two fractions that were in contact with anti-Ago-2 (FIG. 4C; left panel), as expected. Accordingly, the anti-Ago-2-coated beads in contact with both Ago-2 and miR-18a, but not with miR-18a alone, led to the detection of miR-18a by qPCR, because this miRNA was bound to Ago-2 (0.13±0.03; n=3) (FIG. 4C; right panel). The absence of Ago-2 or miR-18a in the isotypic control immunoprecipitated fraction demonstrated that miR-18a was specifically associated with Ago-2.

miRNA delivery reportedly may occur through other mechanisms, namely through exosome release. This hypothesis was excluded by using GW4869, a specific inhibitor of N-Smase-2 (neutral sphingomyelinase-2), necessary for exocytosis. Experiments showed that this agent did not interfere with miR-18a uptake.

### Example 5: Silencing Ago-2 Compromises miR-18a-Induced TSP-1 Increase

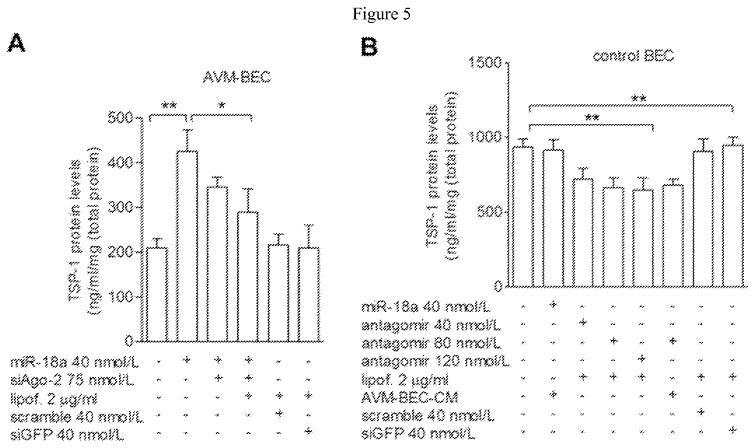

It is reported that miR-18a treatment increases TSP-1 release by AVM-BEC. Hence, we treated AVM-BEC with siAgo-2 (75 nmol/L) to determine if miR-18a-induced TSP-1 release would be decreased in the presence of low Ago-2 levels (FIG. 5A). Thus, AVM-BEC were treated with siAgo-2 and then incubated with miR-18a. The amount of TSP-1 protein secreted by these cells was less than miR-18a treated AVM-BEC (FIG. 5A) (untreated_{AVM-BEC}=209.3±21.12; miR-18a_{AVM-BEC}=425.3±48.3; siAgo2+miR-18a_{AVM-BEC}=289.4±52.34; n=4; p<0.05). In addition, when control BEC were treated with antagomir of miR-18a (40-120 nmol/L), the results showed that inhibition of endogenous miR-18a (80 nmol/L) significantly decreased TSP-1 levels (untreated_{control BEC}=934.8±53.2; lipof.+antagomir80_{conrol BEC}=664.5±66.7; antagomir80+AVM-BEC-CM_{control BEC}=706.7±46.7; n=4; p<0.01) (FIG. 5B). These data show that decreasing Ago-2 decreases miR-18a entry and therefore lowers TSP-1 secretion; thus miR-18a is internalized and is dependent on Ago-2 availability.

### Example 6: Intravenous Administration of miR-18a In Vivo Enhances Anti-Angiogenic Properties

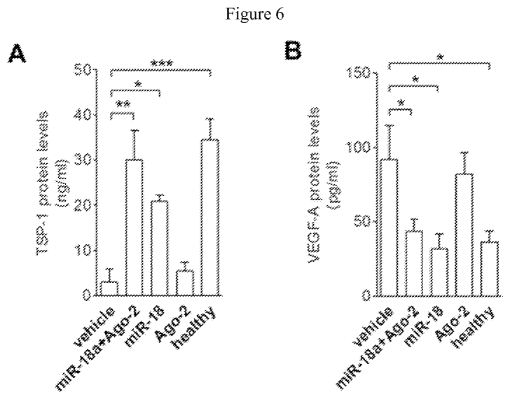

Currently there are no validated in vivo brain AVM models; hence, we used an alternative intracranial tumor model that also exhibited active angiogenesis. To initiate angiogenesis, we injected renilla luciferase-labeled human glioma cells into immune-incompetent athymic nude mice (FIG. 6). In this model, the systemic application of an anti-angiogenic agent has greatest impact when given during the first week post-implantation of tumor cells. Thus, to achieve maximal effects on angiogenesis, treatment was initiated 3 days after intracranial implantation of tumor cells. The animal groups were as follows: group 1: vehicle (PBS); group 2: miR-18a (40 nmol/L) in combination with Ago-2 (0.4 nmol/L); group 3: miR-18 alone (40 nmol); group 4: Ago-2 alone (0.4 nmol/L). Agents were administered intravenously (lateral tail vein) every 48 hours until three treatments were completed per group; animals were then euthanized at day 9 and blood samples were collected. Analysis of blood samples showed that the combination treatment of miR-18a and Ago-2 resulted in a significant change in the key angiogenic factors. TSP-1 and VEGF-A (FIGS. 6A and 6B, respectively). The combination treatment of miR-18a and Ago-2 led to a significant increase in TSP-1 levels as compared to vehicle-treated animals (vehicle=2.9±2.9; miR-18 Ago-2=30.0±6.6; n=5; p<0.01) (FIG. 6A). Furthermore, TSP-1 protein levels in response to miR-18a and Ago-2 co-treatment was similar to levels found in healthy animals (healthy-34.6±4.6; n=3). miR-18a alone also increased TSP-1 levels but to a lesser extent (miR-18a=21.0±1.3; n=5; p<0.05). The combination of miR-18a and Ago-2, or miR-18a alone, led to a decrease in secretion of the pro-angiogenic VEGF-A (vehicle=92.1±22.6; miR-18a+Ago-2=43.6±8.2; miR-18a=32.0±10.1; healthy=36.6±7.3; n=3-5; p<0.05) (FIG. 6B). Ago-2 treatment alone had no effect on either TSP-1 or VEGF-A secretion. Imaging data showed that mice treated with miR-18a alone and in combination with Ago-2 showed a trend towards reduced tumor growth compared to vehicle treatment, although these differences were not significant (p<0.5; p<0.6). To test whether the in vivo data resulted from activation of tumors cells rather than blood vessel cells, we analyzed the levels of miR-18a internalized by the implanted tumor cells (U251). We observed no intracellular uptake of miR-18a over time indicating that the effects of miR-18 and Ago-2 were a reflection of brain endothelial cell internalization and processing and not tumor cell internalization of miR-18a. Based on a histological examination of tissue slides, we did not observe any pathology in peripheral organs in any of the groups tested. Hence, our data show that Ago-2 in combination with miR-18a is effective and functional in vivo.

In this study, we show that AVM-BEC secrete RNA-binding protein Ago-2, which serves as a carrier for miR-18a into brain endothelial cells. Furthermore, the combination of miR-18a and Ago-2 is active in vivo, and can modulate the expression of angiogenic factors, thereby demonstrating that intravascular delivery of miRNA to the brain vasculature is a feasible therapeutic approach.

miRNA is detected extracellularly in a variety of human body fluids including blood. Although the bloodstream is enriched with nucleases, and rapid renal clearance may lead to miRNA degradation and clearance, systemic delivery of miRNA without transfection reagents (naked delivery) has been attempted successfully. Intraperitoneal injection of anti-miR-182 reduced tumor metastasis in the liver of mice, showing that miRNA without transfection reagents can be internalized by tumor cells. Additionally, intravenous injection of anti-miR-122 entered virally infected cells, thus inhibiting viral replication and improving virus-induced liver disease. Mounting evidence has suggested that miRNA, and other nucleic acids. are spared from degradation by either being encased in lipid vesicles, by forming lipoproteins, or forming ribonucleoprotein complexes. Without wishing to be bound by any particular theory, the latter is believed to be main mechanism for miRNA trafficking. In fact, several extracellular miRNAs, including miR-18a, found in human blood plasma or cell culture media are associated with the RNA-binding protein Ago-2.

In mammalian cells, Ago-2 proteins bind intracellularly to endogenous double-stranded small RNAs for incorporation into RISC or extracellularly to exogenous miRNA, such as miR-18a, used in our work. This activity suggests the possibility of a more general mechanism of action for Ago-2 in miRNA delivery. Our results demonstrated that Ago-2 was the major factor in AVM-BEC-CM, responsible for delivery and uptake of miR-18a to AVM-BEC recipient cells, by forming a stable ribonucleoprotein complex with miR-18a. Production of Ago-2 was antagonized in AVM-BEC treated with siAgo-2 (siAgo-2-AVM-BEC-CM) resulting in compromised miR-18a entry and activity (FIGS. 2-5).

These results were further extended by the detection of Ago-2:miR18a complexes internalized by recipient BEC, as demonstrated by confocal fluorescence microscopy (FIG. 3). This analysis clearly showed that the combination of miR-18a with Ago-2 was required for cellular uptake of miR-18a. The addition of AVM-BEC-CM or miR-18a alone did not result in Ago-2 uptake or a functional response. The requirement for this binary complex also implied the specificity of a putative cell membrane receptor for the ribonucleoprotein complex prior to facilitated internalization as shown by fluorescence microscopy (FIG. 4). To the best of our knowledge there is no known ribonucleoprotein receptor for Ago-2 complexes.

Endothelial cells have been shown to use Ago-2 to deliver miRNA cargoes for cell-to-cell communication. Our studies further extend this observation to show that brain endothelial cells are highly permissive for miRNA uptake compared to other endothelial cell types (e.g. HMEC and HUVEC). Whether this is due to an intrinsic property of brain endothelial cells or is related to differences in ribonucleoprotein receptor density among the different endothelial cell types remains to be shown.

Without wishing to be bound by any particular theory, it is believed that miR-18a, through the inhibition of Id-1 expression, derepresses TSP-1 secretion in AVM-BEC, thus “normalizing” the abnormal features of these cells. We demonstrated that miR-18a-induced TSP-1 secretion is compromised in the presence of lower amounts of Ago-2 (FIG. 5), thus corroborating our previous results and extending these observations by demonstrating that Ago-2 was needed for miRNA delivery.

The therapeutic use of Ago-2 as a miRNA carrier bypasses the many challenges faced by in vivo delivery of miRNA, primarily protection from serum nuclease degradation. Since endothelial cells are in direct and exclusive contact with the bloodstream, intravascular miRNA delivery can achieve high target specificity with negligible side effects. An interesting result is that brain endothelial cells are highly permissive for uptake of miR-18a, raising the possibility of intravascular delivery of therapeutic miRNA to AVM-BEC and possibly other brain vascular dysfunctions. As a preliminary demonstration of an in vivo response to intravenous Ago-2 miR-18a delivery we used an intracranial tumor model, which gives rise to highly vascularized tumors (FIG. 6). This approach was used since there are no in vivo brain AVM models. To demonstrate the anti-angiogenic effect of miR-18a and Ago-2 co-treatment, blood samples were collected from Ago-2/miR-18a treated animals to evaluate key angiogenic markers, namely TSP-1 and VEGF-A. miR-18a alone and miR-18a and Ago-2 co-treatment significantly increased TSP-1, and decreased VEGF-A secretion to levels comparable to healthy controls. Administration of miR-18a alone also showed an anti-angiogenic effect; however this could be attributed to the presence of circulating Ago-2 in the bloodstream.

In conclusion, AVM-BEC secrete RNA-binding protein Ago-2 which acts as a carrier for miR-18a to target brain endothelial cells. Moreover, miR-18a delivered by Ago-2 is functionally active both in vitro and in vivo. Thus, Ago-2 can be used as an efficient vehicle for miRNA, supporting the development of safer and more efficient brain endothelial cell-targeted therapies.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

What is claimed is:

1. A method of delivering a miRNA to a cell, comprising:
providing the miRNA and an Argonaute-2 (Ago-2) or a variant thereof; and
contacting the cell with the miRNA and the Ago-2 or the variant thereof, thereby delivering the mRNA to the cell.

2. The method of claim 1, wherein the miRNA and the Ago-2 or the variant thereof are provided in one composition.

3. The method of claim 1, wherein the miRNA and the Ago-2 or the variant thereof are provided in separate compositions.

4. The method of claim 3, wherein the miRNA and the Ago-2 or the variant thereof are mixed prior to contacting the cell with the miRNA and the Ago-2 or the variant thereof.

5. A method of inhibiting angiogenesis, promoting angiogenesis, and/or treating, preventing, reducing the likelihood of having, reducing the severity of and/or slowing the progression of a condition in a subject, comprising:
providing a miRNA and an Argonaute-2 (Ago-2) or a variant thereof;
administering a therapeutically effective amount of the miRNA and the Ago-2 or the variant thereof to the subject, thereby inhibiting angiogenesis, promoting angiogenesis, and/or treating, preventing, reducing the likelihood of having, reducing the severity of and/or slowing the progression of the condition in the subject.

6. The method of claim 5, wherein the miRNA and the Ago-2 or the variant thereof are provided in one composition.

7. The method of claim 5, wherein the miRNA and the Ago-2 or the variant thereof are provided in separate compositions.

8. The method of claim 7, wherein the miRNA and the Ago-2 or the variant thereof are mixed prior to contacting the cell with the miRNA and the Ago-2 or the variant thereof.

9. The method of claim 5, wherein the condition is a neurovascular disease.

10. The method of claim 5, wherein the condition is cerebral arteriovenous malformations (AVM) or stroke.

11. The method of claim 5, wherein the condition is a tumor.

12. The method of claim 5, wherein the condition is brain tumor, glioma, glioblastoma, and/or glioblastoma multiforme (GBM).

13. The method of claim 5, wherein the miRNA is miR-18a or miR-128a.

14. The method of claim 5, wherein the miRNA is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L.

15. The method of claim 5, wherein the Ago-2 or the variant thereof is administered at about 0.001 to 0.01, 0.01 to 0.1, 0.1 to 0.5, 0.5 to 5, 5 to 10, 10 to 20, 20 to 50, 50 to 100, 100 to 200, 200 to 300, 300 to 400, 400 to 500, 500 to 600, 600 to 700, 700 to 800, 800 to 900, or 900 to 1000 nmol/L.

16. The method of claim 5, wherein the miRNA and/or the Ago-2 or the variant thereof are administered intratumorally, intracranially, intraventricularly, intrathecally, epidurally, intradurally, intravascularly, intravenously, intraarterially, intramuscularly, subcutaneously, intraperitoneally, intranasally, or orally.

17. The method of claim 5, wherein the miRNA and/or the Ago-2 or the variant thereof are administered once, twice, three or more times.

18. The method of claim 5, wherein the miRNA and/or the Ago-2 or the variant thereof are administered 1-3 times per day, 1-7 times per week, or 1-9 times per month.

19. The method of claim 5, further comprising providing and administering a therapeutically effective amount of an anti-angiogenic drug to the subject.

20. The method of claim 5, further comprising providing and administering a therapeutically effective amount of a chemotherapeutic agent to the subject.

21. A kit, comprising:
a quantity of a miRNA;
a quantity of an Argonaute-2 (Ago-2) or a variant thereof; and
instructions for using the Ago-2 or the variant thereof to deliver the miRNA, to inhibit angiogenesis, to promote angiogenesis, and/or to treat, prevent, reduce the likelihood of having, reduce the severity of and/or slow the progression of a condition in a subject.

22. A composition comprising a miRNA and an Argonaute-2 (Ago-2) or a variant thereof.

23. The composition of claim 22, wherein the miRNA is miR-18a or miR-128a.

24. The composition of claim 22, wherein the composition comprises a ribonucleoprotein complex of the miRNA and the Ago-2 or the variant thereof.

25. The composition of claim 22, further comprising an anti-angiogenic drug.

26. The composition of claim 22, further comprising a chemotherapeutic agent.

27. The composition of claim 22, wherein the composition is formulated for intratumoral, intracranial, intraventricular, intrathecal, epidural, intradural, intravascular, intravenous, intraarterial, intramuscular, subcutaneous, intraperitoneal, intranasal, or oral administration
